(19) United States
(12) Patent Application Publication
(10) Pub. No.: US 2016/0166174 A1
(43) Pub. Date: Jun. 16, 2016

(54) SYSTEM AND METHOD FOR REAL TIME TRACKING AND MODELING OF SURGICAL SITE

(71) Applicant: Navigate Surgical Technologies, Inc., Vancouver (CA)

(72) Inventors: Ehud (Udi) DAON, North Vancouver (CA); Martin Gregory BECKETT, Bowen Island (CA); Vili PETEK, Vancouver (CA)

(21) Appl. No.: 15/048,772

(22) Filed: Feb. 19, 2016

Related U.S. Application Data
(63) Continuation-in-part of application No. 14/645,927, filed on Mar. 12, 2015.
Application No. 14/645,927 is a continuation-in-part of application No. 14/599,149, filed on Jan. 16, 2015.
Application No. 14/599,149 is a division of application No. 13/571,285, filed on Aug. 9, 2012.
Application No. 13/571,285 is a division of application No. 13/571,284, filed on Aug. 9, 2012, now Pat. No. 8,938,282.
Application No. 13/571,284 is a continuation-in-part of application No. 13/822,358, filed on Mar. 12, 2013.
(60) Provisional application No. 61/952,832, filed on Mar. 13, 2014.
(60) Provisional application No. 61/553,058, filed on Oct. 28, 2011.
(60) Provisional application No. 61/671,861, filed on Jul. 16, 2012.

(51) Int. Cl. A61B 5/06; A61B 5/00; A61B 17/3211; A61F 2/02; A61C 3/02; A61B 1/00

(52) U.S. Cl. CPC A61B 5/064; A61C 3/02; A61B 1/00; A61B 17/3211; A61F 2/02; A61B 5/0022; A61B 2034/2055

(57) ABSTRACT

A surgical site monitoring system and associated method of use employ a single scan-visible passive vectorized fiducial reference attached at a fiducial location near a surgical site. A passive vectorized tracking marker is attached to the fiducial reference. Passive vectorized tracking markers may also be attached to surgical implements used at the surgical site. A scan prior to a surgical procedure with the fiducial reference attached is used to obtain scan data of the surgical site. A tracker obtains image information about the tracking markers and surgical implements. The system and method employ the scan data and image information to track the surgery and the surgical implements relative to the surgical site. The system and method use either markings on or shapes of the tracking markers to determine from the image information the relative 3D locations and orientations of the surgical implements and fiducial reference. In some embodiments, changes in the surgical implements, for example drill bit changes, are also tracked during surgical procedures.

### CROSS-REFERENCE TO RELATED APPLICATIONS

The present application is a Continuation-in-part of U.S. patent application Ser. No. 14/645,927 which claims priority under 35 U.S.C. §119(e) of U.S. Provisional Patent Application Ser. No. 61/952,832, filed Mar. 13, 2014; and is a continuation-in-part of U.S. patent application Ser. No. 14/599,149, filed Jan. 16, 2015, which is a divisional application of U.S. patent application Ser. No. 13/571,284, filed Aug. 9, 2012, and also claims priority to U.S. patent application Ser. No. 13/822,358, filed Mar. 12, 2013, both of which claim priority under 35 U.S.C. §119(e) of U.S. Provisional Patent Application Ser. Nos. 61/553,058, filed Oct. 28, 2011, and 61/616,718, filed Mar. 28, 2012.

### BACKGROUND OF THE INVENTION

1. Field of the Invention

The invention relates to location monitoring hardware and software systems. More specifically, the field of the invention is that of surgical equipment and software for monitoring surgical conditions.

2. Description of the Related Art

Visual and other sensory systems are known, with such systems being capable of both observing and monitoring surgical procedures. With such observation and monitoring systems, computer aided surgeries are now possible, and in fact are being routinely performed. In such procedures, the computer software interacts with both clinical images of the patient and observed surgical images from the current surgical procedure to provide guidance to the physician in conducting the surgery. For example, in one known system a carrier assembly bears at least one fiducial marker onto an attachment element in a precisely repeatable position with respect to a patient's jaw bone, employing the carrier assembly for providing registration between the fiducial marker and the patient's jaw bone and implanting the tooth implant by employing a tracking system which uses the registration to guide a drilling assembly. With this relatively new computer implemented technology, further improvements may further advance the effectiveness of surgical procedures.

### SUMMARY OF THE INVENTION

The present invention involves embodiments of surgical hardware and software monitoring system and method which allows for surgical planning while the patient is available for surgery, for example while the patient is being prepared for surgery so that the system may model the surgical site. In one embodiment, the model may be used to track contemplated surgical procedures and warn the physician regarding possible boundary violations that would indicate an inappropriate location in a surgical procedure. In another embodiment, the hardware may track the movement of instruments during the procedure and in reference to the model to enhance observation of the procedure. In this way, physicians are provided an additional tool to improve surgical planning and performance

The system uses a particularly configured passive vectorized fiducial reference, to orient the monitoring system with regard to the critical area. The fiducial reference is attached to a location near the intended surgical area. For example, in the example of a dental surgery, a splint may be used to securely locate the fiducial reference near the surgical area. The fiducial reference may then be used as a point of reference, or a fiducial, for the further image processing of the surgical site. The fiducial reference may be identified relative to other portions of the surgical area by having a recognizable fiducial marker apparent in the scan.

The embodiments of the invention involve automatically computing the three-dimensional location of the patient by means of a tracking device that may be a passive vectorized tracking marker. The tracking marker may be attached in fixed spatial relation either directly to the fiducial reference, or attached to the fiducial reference via a tracking pole that itself may have a distinct three-dimensional shape. In the dental surgery example, a tracking pole is mechanically connected to the base of the fiducial reference that is in turn fixed in the patient's mouth. Each tracking pole device has a particular observation pattern, located either on itself or on a suitable passive vectorized tracking marker, and a particular geometrical connection to the base, which the computer software recognizes as corresponding to a particular geometry for subsequent location calculations. Although individual tracking pole devices have distinct configurations, they may all share the same connection base and thus may be used with any passive vectorized fiducial reference. The particular tracking information calculations are dictated by the particular tracking pole used, and actual patient location is calculated accordingly. Thus, tracking pole devices may be interchanged and calculation of the location remains the same. This provides, in the case of dental surgery, automatic recognition of the patient head location in space. Alternatively, a sensor device, or a tracker, may be in a known position relative to the fiducial key and its tracking pole, so that the current data image may be mapped to the scan image items.

The vectorized fiducial reference and each tracking pole or associated passive vectorized tracking marker may have a pattern made of radio opaque material so that when imaging information is scanned by the software, the particular items are recognized. Typically, each instrument used in the procedure has a unique pattern on its associated tracking marker so that the tracker information identifies the instrument. The software creates a model of the surgical site, in one embodiment a coordinate system, according to the location and orientation of the patterns on the fiducial reference and/or tracking pole(s) or their attached tracking markers. By way of example, in the embodiment where the fiducial reference has an associated pre-assigned pattern, analysis software interpreting image information from the tracker may recognize the pattern and may select the site of the base of the fiducial to be at the location where the fiducial reference is attached to a splint. If the fiducial key does not have an associated pattern, a fiducial site is designated. In the dental example this can be at a particular spatial relation to the tooth, and a splint location can be automatically designed for placement of the fiducial reference.

The system may further comprise a surgical implement bearing a third passive vectorized tracking marker, wherein the tracker is further configured and disposed for obtaining image information of the third tracking marker; the software program has a further series of instructions which when executed by the processor determines from the image information the current position and orientation of the third tracking marker and relates the scan data to the current position and orientation of the surgical implement.

In a further aspect, a system is provided for monitoring in three dimensions relative to a surgical site changes in a surgical implement that comprises an interchangeable portion and an invariant portion, the system comprising: a single passive vectorized scan-visible fiducial reference configured for attaching to a fiducial location proximate the surgical site; a first passive vectorized tracking marker disposable in fixed spatial relation with the fiducial reference; a second passive vectorized tracking marker rigidly attached to the invariant portion of the surgical instrument in a known location and orientation relative to the invariant portion; a tracker disposed to obtain image information from a field of view including at least the first tracking marker and the surgical implement; a controller having pre-surgical scan data from a scan of the surgical site and the fiducial location with the fiducial reference attached at the fiducial location, the controller configured to receive the image information from the tracker and including a processor with memory and a software program having a series of instructions which when executed by the processor determines from the image information a three-dimensional location and orientation of the first tracking marker relative to the surgical site, identifies the interchangeable portion of the surgical implement in the image information, and determines from the image information and from the three-dimensional location and orientation of the first tracking marker relative to the surgical site a three-dimensional location and orientation of a working tip of the interchangeable portion of the surgical implement relative to the surgical sit. The tracker may be a non-stereo optical tracker.

The software may comprise a further series of instructions which when executed determines from the image information the three-dimensional location and orientation of the second tracking marker. The system may further comprise a database of pre-surgical information of the implement and wherein the software comprises a further series of instructions which when executed indentifies the interchangeable portion of the surgical implement in the image information based on the pre-surgical information in the database. In other embodiments, the software may comprise a further series of instructions which when executed determines the three dimensional location of the working tip of the interchangeable portion and a series of instructions which when executed determines the length of the interchangeable portion from the three-dimensional location of the working tip and the three-dimensional location and orientation of the second tracking marker and the invariant portion attached to the second tracking marker. In yet other embodiments, the software may comprise a series of instructions which when executed triangulates the three dimensional location of the working tip based on two separate perspectives of the interchangeable portions in the field of view of the tracker.

In another aspect, a method is provided for monitoring changes in a surgical implement in three dimensions relative to a surgical site, the method comprising: attaching a single passive vectorized scan-visible fiducial reference at a fiducial location proximate the surgical site; obtaining scan data by performing a scan of the surgical site and the fiducial location with the fiducial reference attached; obtaining from the scan data a three-dimensional spatial relationship between the fiducial reference and the surgical site; disposing in a field of view of a tracker a first passive vectorized tracking marker in fixed spatial relation with the fiducial reference; disposing in the field of view of the tracker the surgical implement comprising an interchangeable portion and an invariant portion, the invariant portion bearing a second passive vectorized marker; obtaining image information of the field of view from the tracker; determining from the image information a three-dimensional location and orientation of the first tracking marker relative to the surgical site; identifying the interchangeable portion of the surgical implement in the image information; and determining from the image information and from the three-dimensional location and orientation of the first tracking marker relative to the surgical site the three-dimensional location and orientation of a working tip of the interchangeable portion of the surgical implement relative to the surgical site.

The determining of the location and orientation of a working tip of the interchangeable portion relative to the surgical site may comprise determining from the image information the three-dimensional location and orientation of the second tracking marker attached to the invariant portion. The identifying the interchangeable portion of the surgical implement in the image information may be based on pre-surgical information in a database. In other embodiments, the identifying the interchangeable portion of the surgical implement in the image information may comprises determining the three dimensional location of the working tip of the interchangeable portion and determining the length of the interchangeable portion from the three-dimensional location of the working tip and the three-dimensional location and orientation of the second tracking marker and the invariant portion attached to the second tracking marker. In yet further embodiments, the determining the three dimensional location of a working tip of the interchangeable portion may comprise triangulating the three dimensional location of the working tip based on two separate perspectives of the interchangeable portions in the field of view of the tracker.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized as controller and display in conjunction with embodiments of the present invention.

FIGS. 3A-J are drawings of hardware components of the surgical monitoring system according to embodiments of the invention.

FIGS. 4A-C is a flowchart diagram illustrating one embodiment of the registering method of the present invention.

FIG. 5 is a drawing of a passive vectorized dental fiducial key with a tracking pole and a dental drill according to one embodiment of the present invention.

FIG. 6 is a drawing of a surgical site showing a passive vectorized fiducial key, endoscope, and biopsy needle according to another embodiment of the invention.

FIGS. 7 is a flow chart of a method for relating in real time a three-dimensional location and orientation of a surgical site according to the present invention

FIG. 8 is a flow chart of a method for real time monitoring of the position of a surgical implement in relation to a surgical site according to the present invention

FIGS. 9A and 9B are more detailed drawings of an element of FIG. 5.

FIG. 10 is a flow chart of a method for monitoring changes in a surgical implement according to the present invention.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

The detailed descriptions that follow are presented in part in terms of algorithms and symbolic representations of operations on data bits within a computer memory representing alphanumeric characters or other information. The hardware components are shown with particular shapes and relative orientations and sizes using particular scanning techniques, although in the general case one of ordinary skill recognizes that a variety of particular shapes and orientations and scanning methodologies may be used within the teaching of the present invention. A computer generally includes a processor for executing instructions and memory for storing instructions and data, including interfaces to obtain and process imaging data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities, observing and measuring scanned data representative of matter around the surgical site. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed to capture the underlying data of an image. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements that impart or manifest a physical organization on the information stored in memory. More than mere abstraction, the data structures are specific electrical or magnetic structural elements in memory, which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein that form part of the present invention; the operations are machine operations. Useful machines for performing the operations of the present invention include general-purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

The present invention also relates to an apparatus for performing these operations. This apparatus may be specifically constructed for the required purposes or it may comprise a general-purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipments through signals configured to particular protocols, which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects. Often, but not necessarily, a physical object has a corresponding software object that may collect and transmit observed data from the physical device to the software system. Such observed data may be accessed from the physical object and/or the software object merely as an item of convenience; therefore where “actual data” is used in the following description, such “actual data” may be from the instrument itself or from the corresponding software object or module.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and here the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions, which may include sending additional messages to one or more other objects. The other objects may in turn carry out additional functions in response to the messages they receive. Including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms that are used frequently have specialized meanings in the present context. The term “object” relates to a set of computer instructions and associated data, which may be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers that are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. Examples of Browsers compatible with the present invention include the Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information, which is formatted in a Standard Generalized Markup Language (“SGML”) or a HyperText Markup Language (“HTML”), both being scripting languages, which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular, or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD″) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system, which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Washington), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.) , and Windows Phone 7. “Mobile Apps” refers to software programs written for execution with Mobile Software.

The terms “scan, fiducial reference”, “fiducial location”, “marker,” “tracker” and “image information” have particular meanings in the present disclosure. For purposes of the present disclosure, “scan” or derivatives thereof refer to x-ray, magnetic resonance imaging (MRI), computerized tomography (CT), sonography, cone beam computerized tomography (CBCT), or any system that produces a quantitative spatial representation of a patient and a “scanner” is the means by which such scans are obtained. The term “fiducial key”, or “fiducial reference”, or simply “fiducial” refers to an object or reference on the image of a scan that is uniquely identifiable as a fixed recognizable point. In the present specification the term “fiducial location” refers to a useful location to which a fiducial reference is attached. A “fiducial location” will typically be proximate a surgical site. The term “marker” or “tracking marker” refers to an object or reference that may be perceived by a sensor proximate to the location of the surgical or dental procedure, where the sensor may be an optical sensor, a radio frequency identifier (RFID), a sonic motion detector, an ultra-violet or infrared sensor. The term “tracker” refers to a device or system of devices able to determine the location of the markers and their orientation and movement continually in ‘real time’ during a procedure. As an example of a possible implementation, if the markers are composed of printed targets then the tracker may include a stereo camera pair. In some embodiments, the tracker may be a non-stereo optical tracker, for example a camera. The camera may, for example, operate in the visible or near-infrared range. The term “image information” is used in the present specification to describe information obtained by the tracker, whether optical or otherwise, and usable for determining the location of the markers and their orientation and movement continually in ‘real time’ during a procedure. In some embodiments, an imaging device may be employed to obtain real time close-up images of the surgical site quite apart from the tracker. In this specification, such imaging devices are described by the term “in situ imager” and the in situ imager may comprise an “illuminator” and an “imaging sensor”. The term “vectorized” is used in this specification to describe fiducial keys and tracking markers that are at least one of shaped and marked so as to make their orientation in three dimensions uniquely determinable from their appearance in a scan or in image information. If their three-dimensional orientation is determinable, then their three-dimensional location is also known.

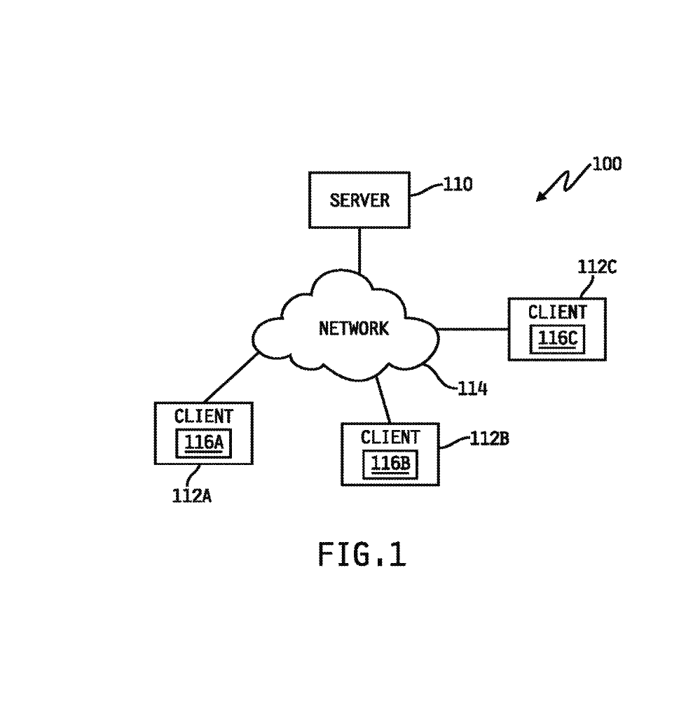

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

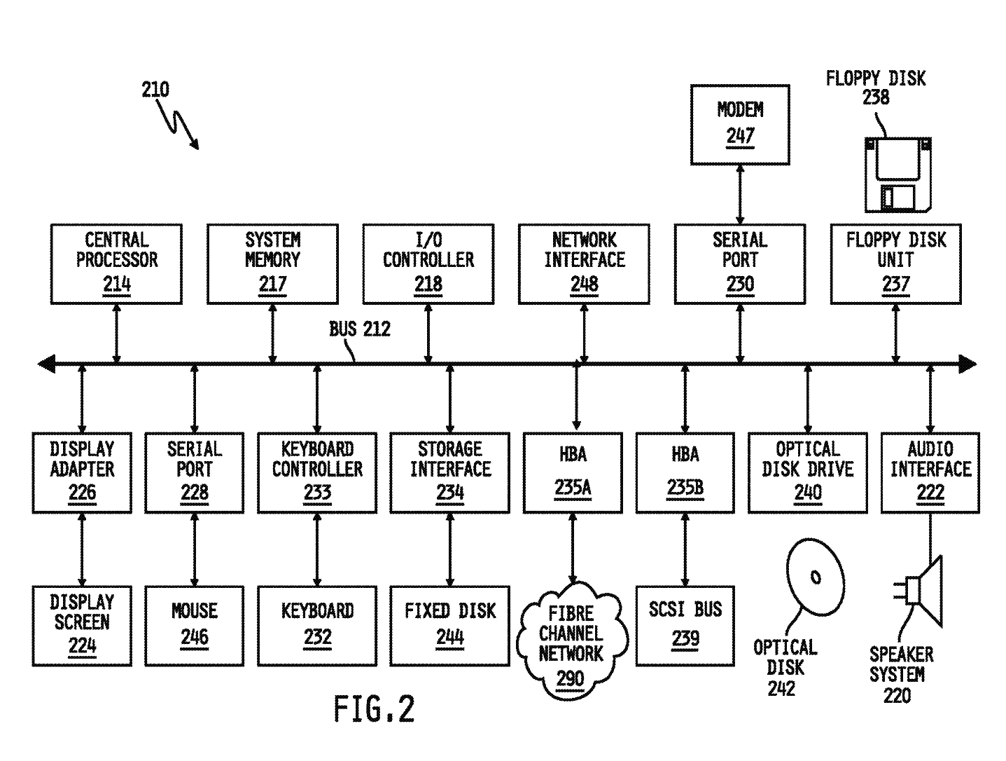

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fiber Channel network 290, host bus adapter (HBA) interface card 235B operative to connect to SCSI bus 239, and optical disk drive 240 operative to receive optical disk 242. Also included are mouse 246 (or other point-and-click device. coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS), which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an Internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

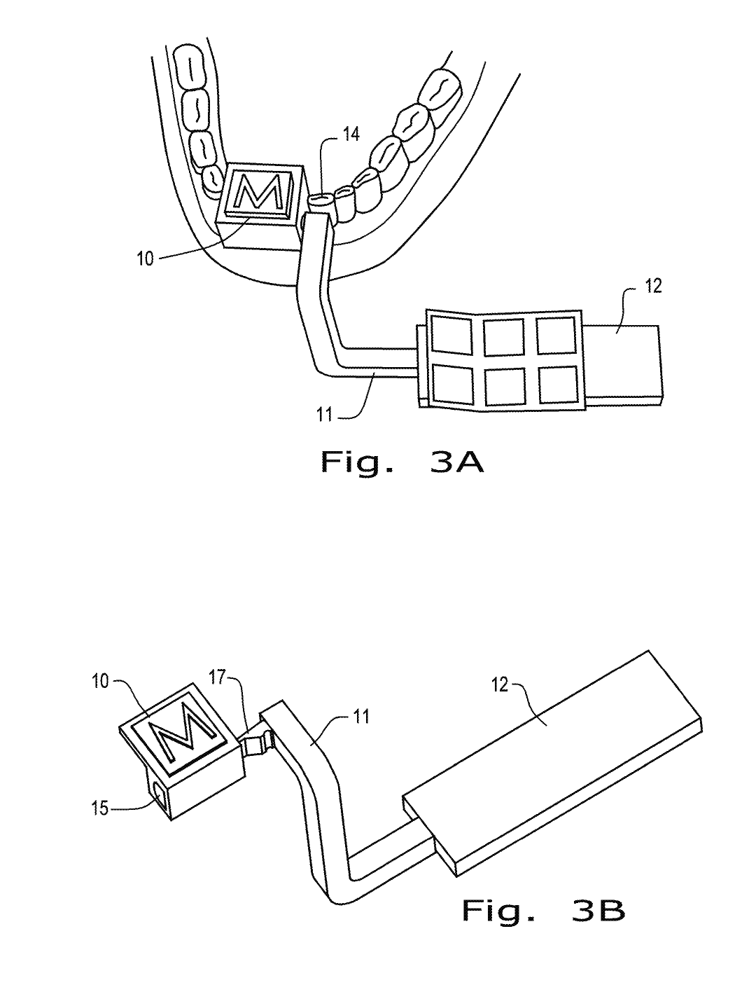

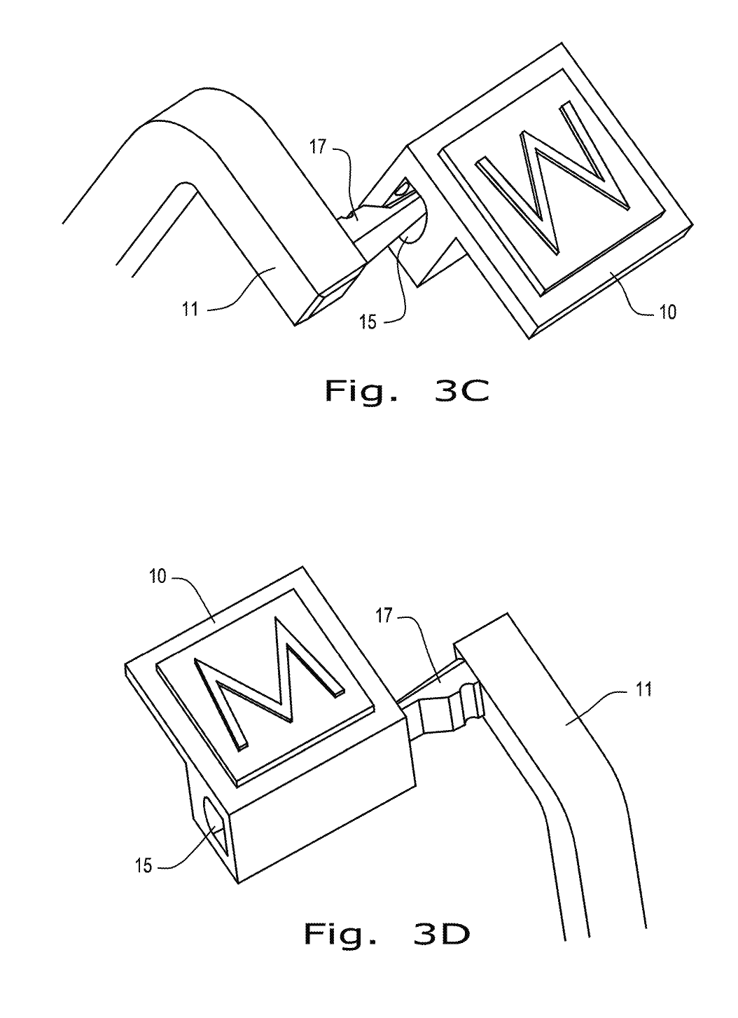

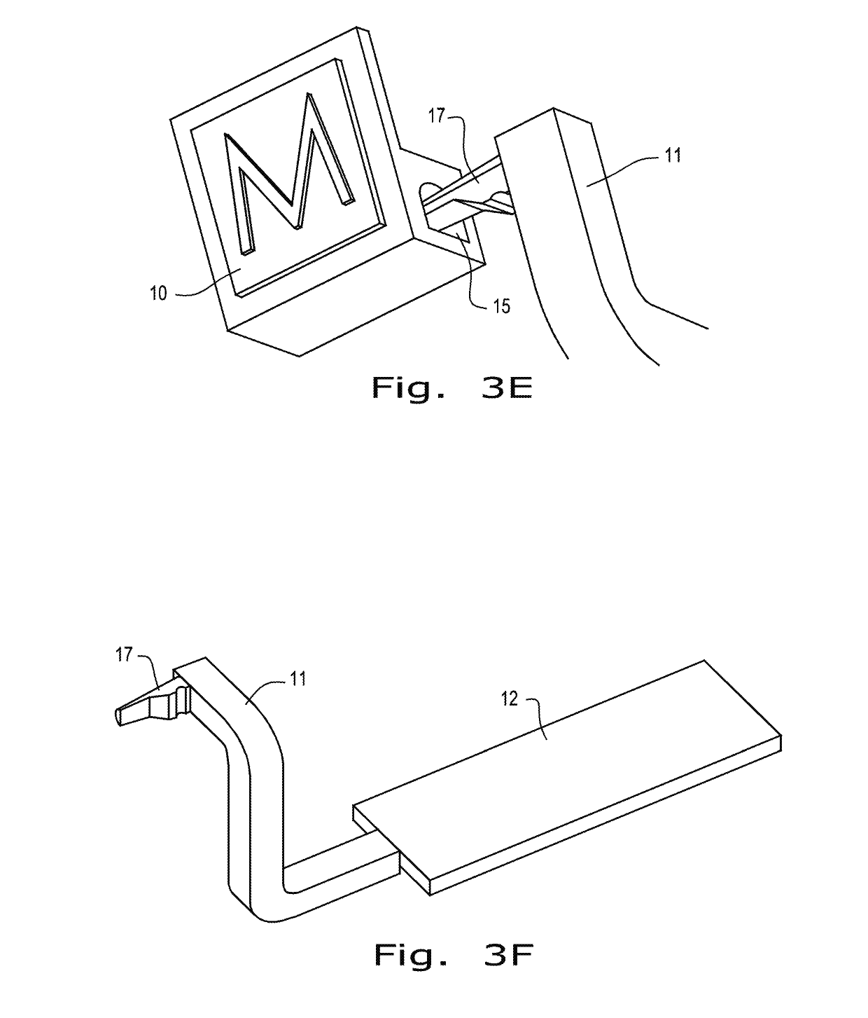

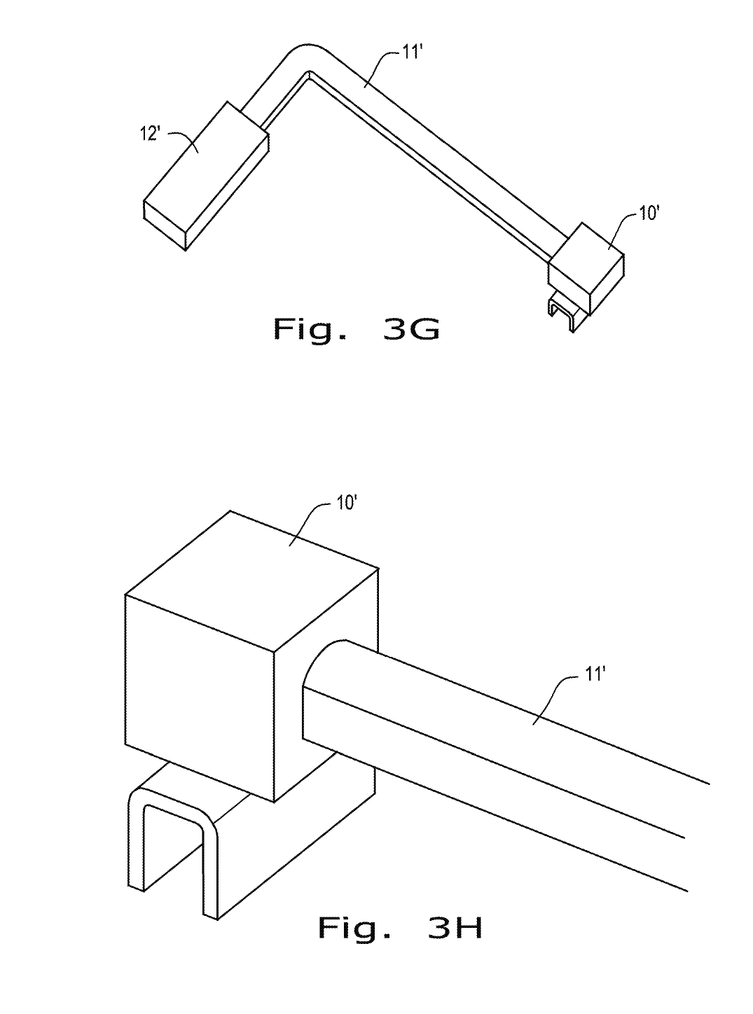

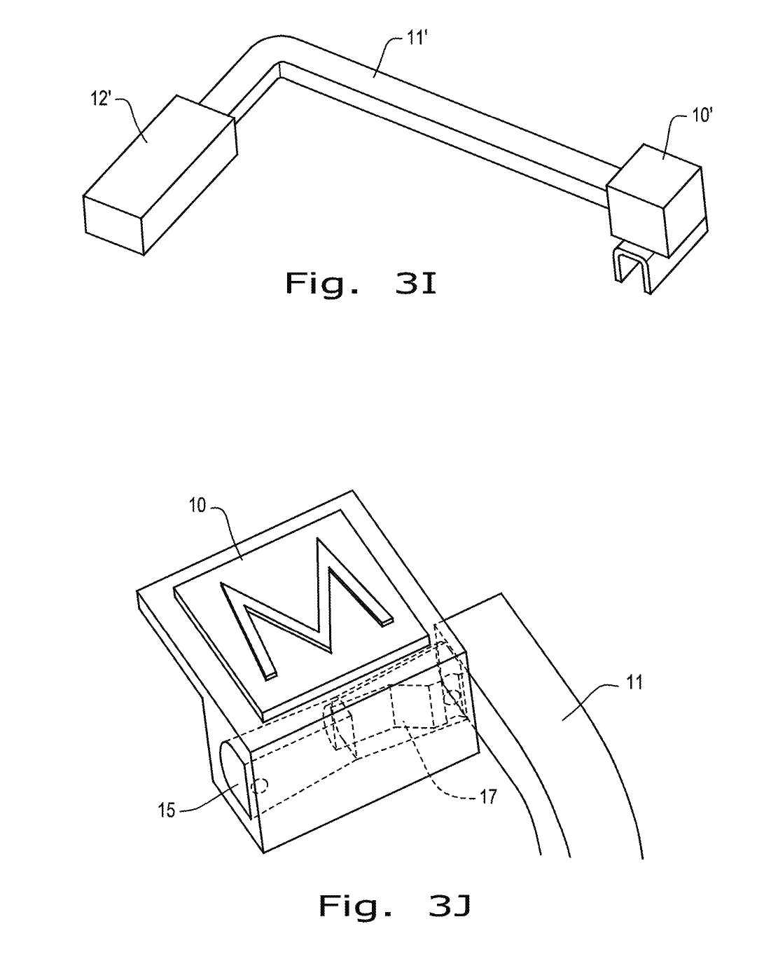

Many other devices or subsystems (not shown) may be connected in a similar manner (e. g., document scanners, digital cameras and so on), including the hardware components of FIGS. 3A-M, which alternatively may be in communication with associated computational resources through local, wide-area, or wireless networks or communications systems. Thus, while the disclosure may generally discuss an embodiment where the hardware components are directly connected to computing resources, one of ordinary skill in this area recognizes that such hardware may be remotely connected with computing resources. Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Washington), WINDOWS® (WINDOWS is a registered trademark of Microsoft Corporation of Redmond, Washington), OS/2® (OS/2 is a registered trademark of International Business Machines Corporation of Armonk, N.Y.), UNIX® (UNLX is a registered trademark of X/Open Company Limited of Reading, United Kingdom), Linux® (Linux is a registered trademark of Linus Torvalds of Portland, Oreg.), or other known or developed operating system.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above-described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modification to the first signal, whether due to circuit limitations or due to passage through other circuit elements which do not change the informational and/or final functional aspect of the first signal.

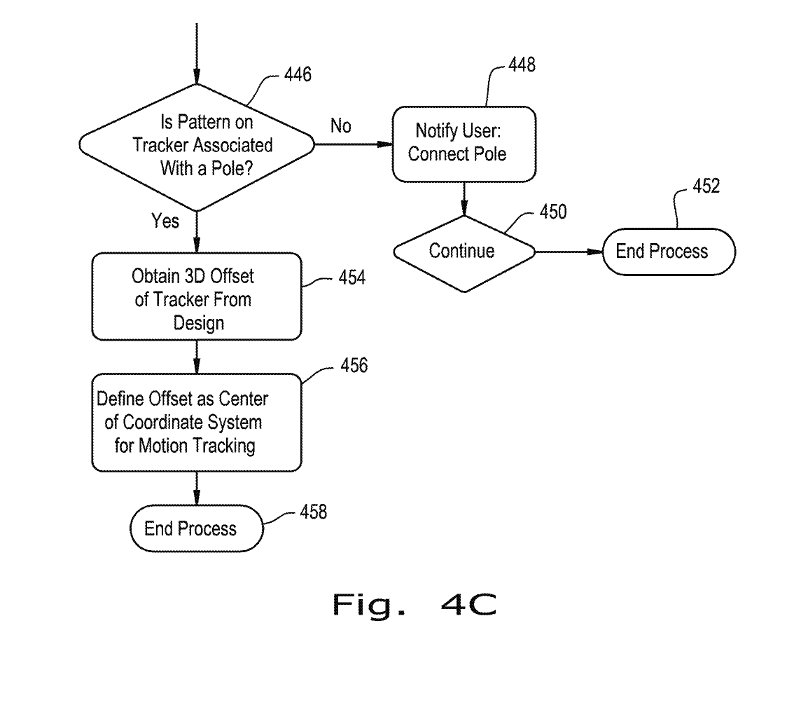

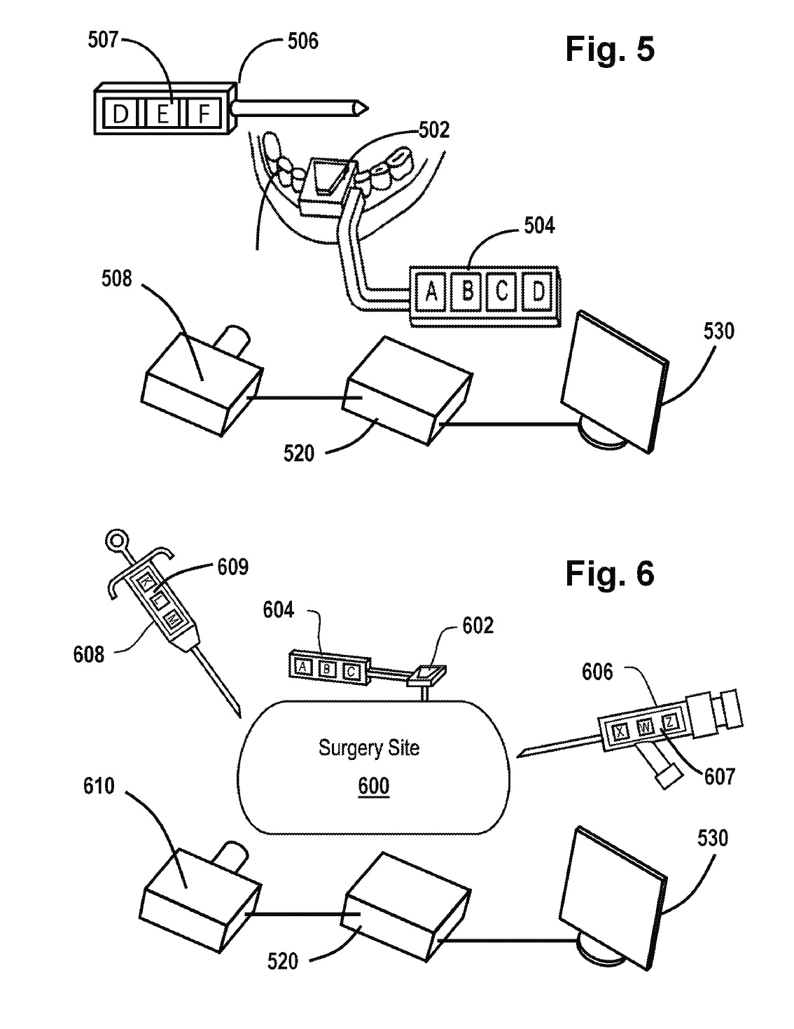

The present invention relates to embodiments of surgical hardware and software monitoring systems and methods which allow for surgical planning while the patient is available for surgery, for example while the patient is being prepared for surgery so that the system may model the surgical site. The system uses a particularly configured piece of hardware, namely a vectorized fiducial reference, represented as fiducial key 10 in FIG. 3A, to orient vectorized tracking marker 12 of the monitoring system with regard to the critical area of the surgery. Single fiducial key 10 is attached to a location near the intended surgical area, in the exemplary embodiment of the dental surgical area of FIG. 3A, fiducial key 10 is attached to a dental splint 14. Vectorized tracking marker 12 may be connected to fiducial key 10 by tracking pole 11. In embodiments in which the fiducial reference is directly visible to a suitable tracker (see for example FIG. 5 and FIG. 6) that acquires image information about the surgical site, a tracking marker may be attached directly to the fiducial reference. The tracker may be in some embodiments a non-stereo optical tracker. For example, in a dental surgical procedure, the dental tracking marker 14 may be used to securely locate the fiducial 10 near the surgical area. The single fiducial key 10 may be used as a point of reference, or a fiducial, for the further image processing of data acquired from tracking marker 12 by the tracker. In this arrangement, fiducial key or reference 10 is scanned not by the tracker, which may for example be an optical tracker, either stereo or non-stereo, but by a suitable scanning means, which may for example be an X-ray system, CAT scan system, or MRI system as per the definition of “scan” above. In some applications, fiducial key 10 may be disposed in a location or in such orientation as to be at least in part non-visible to the tracker of the system.

In other embodiments additional vectorized tracking markers 12 may be attached to items independent of the fiducial key 10 and any of its associated tracking poles 11 or tracking markers 12. This allows the independent items to be tracked by the tracker.

In a further embodiment at least one of the items or instruments near the surgical site may optionally have a tracker attached to function as tracker for the monitoring system of the invention and to thereby sense the orientation and the position of the tracking marker 12 and of any other additional vectorized tracking markers relative to the scan data of the surgical area. By way of example, the tracker attached to an instrument may be a miniature digital camera and it may be attached, for example, to a dentist's drill. Any other vectorized markers to be tracked by the tracker attached to the item or instrument must be within the field of view of the tracker.

Using the dental surgery example, the patient is scanned to obtain an initial scan of the surgical site. The particular configuration of single fiducial key 10 allows computer software stored in memory and executed in a suitable controller, for example processor 214 and memory 217 of computer 210 of FIG. 2, to recognize its relative position within the surgical site from the scan data, so that further observations may be made with reference to both the location and orientation of fiducial key 10. In some embodiments, the fiducial reference includes a marking that is apparent as a recognizable identifying symbol when scanned In other embodiments, the fiducial reference includes a shape that is distinct in the sense that the body apparent on the scan has an asymmetrical form allowing the front, rear, upper, and lower, and left/right defined surfaces that may be unambiguously determined from the analysis of the scan, thereby to allow the determination not only of the location of the fiducial reference, but also of its orientation. That is, the shape and/or markings of the fiducial reference render it vectorized. The marking and/or shape of fiducial key 10 allows it to be used as the single and only fiducial key employed in the surgical hardware and software monitoring system. By comparison, prior art systems typically rely on a plurality of fiducials. Hence, while the tracker may track several vectorized tracking markers within the monitoring system, only a single vectorized fiducial reference or key 10 of known shape or marking is required. By way of example, FIG. 5, later discussed in more detail, shows vectorized marker 504, as well as a vectorized marker 507 on implement 506 being tracked by tracker 508, but there is only one vectorized fiducial reference or key 502 in the system. FIG. 6 similarly shows vectorized marker 604, as well as vectorized markers 607 and 609 on implements 606 and 608 respectively being tracked by tracker 610, while there is only a single vectorized fiducial reference or key 602 in the system.

In addition, the computer software may create a coordinate system for organizing objects in the scan, such as teeth, jaw bone, skin and gum tissue, other surgical instruments, etc. The coordinate system relates the images on the scan to the space around the fiducial and locates the instruments bearing markers both by orientation and position. The model generated by the monitoring system may then be used to check boundary conditions, and in conjunction with the tracker display the arrangement in real time on a suitable display, for example display 224 of FIG. 2 or monitor 530 in FIGS. 5 and 6.

In one embodiment, the computer system has a predetermined knowledge of the physical configuration of single fiducial key 10 and examines slices/sections of the scan to locate fiducial key 10. Locating of fiducial key 10 may be on the basis of its distinct shape, or on the basis of distinctive identifying and orienting markings upon the fiducial key or on attachments to fiducial key 10 such as tracking marker 12. Fiducial key 10 may be rendered distinctly visible in the scans through higher imaging contrast by the employ of radio-opaque materials or high-density materials in the construction of the fiducial key 10. In other embodiments the material of the distinctive identifying and orienting markings may be created using suitable high density or radio-opaque inks or materials. In the present specification, the term “scan-visible” is used to describe the characteristic of fiducial key 10 by which it is rendered visible in a scan, while not necessarily otherwise visible to the human eye or optical sensor.

Once fiducial key 10 is identified, the location and orientation of the fiducial key 10 is determined from the scan segments, and a point within fiducial key 10 is assigned as the center of the coordinate system. The point so chosen may be chosen arbitrarily, or the choice may be based on some useful criterion. A model is then derived in the form of a transformation matrix to relate the fiducial system, being fiducial key 10 in one particular embodiment, to the coordinate system of the surgical site. The resulting virtual construct may be used by surgical procedure planning software for virtual modeling of the contemplated procedure, and may alternatively be used by instrumentation software for the configuration of the instrument, for providing imaging assistance for surgical software, and/or for plotting trajectories for the conduct of the surgical procedure.

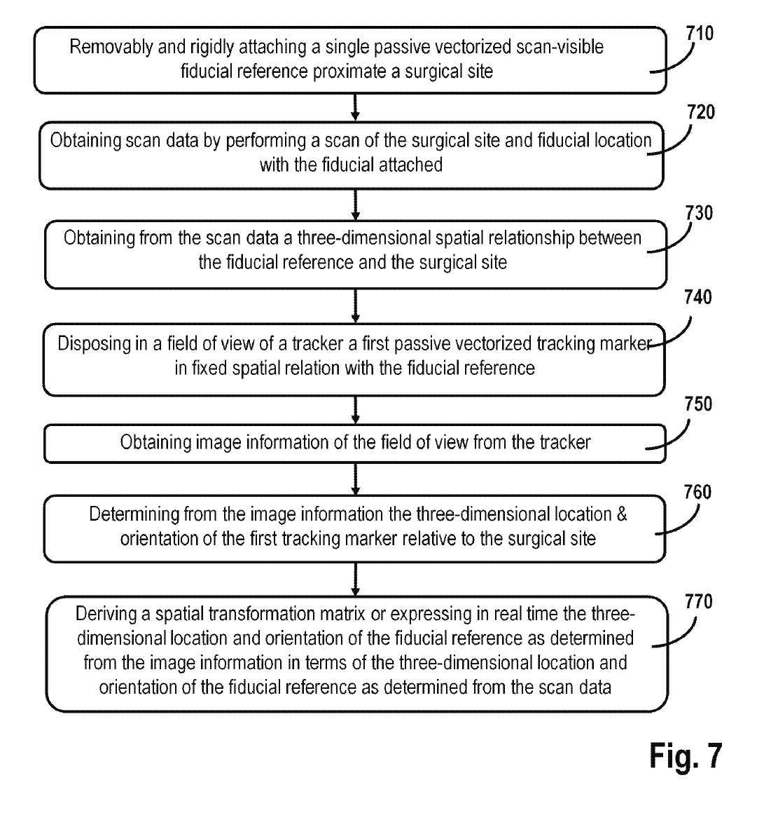

In some embodiments, the monitoring hardware includes a tracking attachment to the fiducial reference. In the embodiment pertaining to dental surgery the tracking attachment to fiducial key 10 is tracking marker 12, which is attached to fiducial key 10 via tracking pole 11. Tracking marker 12 may have a particular identifying pattern, described in more detail later at the hand of FIGS. 7-10. The trackable attachment, for example tracking marker 12, and even associated tracking pole 11 may have known configurations so that observational data from tracking pole 11 and/or tracking marker 12 may be precisely mapped to the coordinate system, and thus progress of the surgical procedure may be monitored and recorded. Further, tracking pole 11 may be used to place tracking marker 12 in such a position that unobstructed access to the surgical or dental operation site is available rather than tracking marker 12 being in a position to obscure a user's access. For example, as particularly shown in FIG. 3J, fiducial key 10 may have hole 15 in a predetermined location specially adapted for engagement with insert 17 of tracking pole 11. In such an arrangement, for example, tracking poles 11 may be attached with a low force push into hole 15 of fiducial key 10, and an audible haptic notification may thus be given upon successful completion of the attachment. In other embodiments, the tracking marker may be adaptable in its physical arrangement to permit unobstructed access to a surgical site during different procedures or stages of a surgical procedure.

It is further possible to reorient the tracking pole during a surgical procedure. Such reorientation may be in order to change the location of the procedure, for example where a dental surgery deals with teeth on the opposite side of the mouth, where a surgeon switches hands, and/or where a second surgeon performs a portion of the procedure. For example, the movement of the tracking pole, for example to allow access to a particular part of the surgical site, may trigger a re-registration of the tracking pole with relation to the coordinate system, so that the locations may be accordingly adjusted. Such a re-registration may be automatically initiated when, for example in the case of the dental surgery embodiment, tracking pole 11 with its attached tracking marker 12 are removed from hole 15 of fiducial key 10 and another tracking marker with its associated tracking pole is connected to an alternative hole on fiducial key 10. Additionally, boundary conditions may be implemented in the software so that the user is notified when observational data approaches and/or enters the boundary areas.

In a further embodiment, the tracking markers may specifically have an identifiably unique three dimensional shape. Suitable three-dimensional shapes may include, without limitation, a segment of an ellipsoid surface and a segment of a cylindrical surface. In general, suitable three-dimensional shapes are shapes that are mathematically describable by simple functions. The tracking markers may bear suitable identifying patterns. The tracking marker may have a distinct sensible characteristic which is identifiable in the image information. For example, without limitation, tracking marker may have a sensible characteristic, that is which is identifiable in the image information, by a specific code or graphic design embedded or imprinted upon its surface that is determinable from the image information. Each specific code may be used to indicate a specific marker, including but not limited to, a specific marker of which a position and orientation relative to the fiducial reference is known and fixed. Such identifiable shapes, patterns, sensible characteristics, distinct characteristics and codes may be stored in a computer data base along with other relevant information including association and position information.

In a further embodiment of the system utilizing the invention, a surgical instrument or implement, herein termed a “hand piece” (see FIGS. 5 and 6), may also have a particular configuration that may be located and tracked in the coordinate system and may have suitable tracking markers as described herein. A boundary condition may be set up to indicate a potential collision with virtual material, so that when the hand piece is sensed to approach the boundary condition an indication may appear on a screen, or an alarm sound. Further, target boundary conditions may be set up to indicate the desired surgical area, so that when the trajectory of the hand piece is trending outside the target area an indication may appear on screen or an alarm sound indicating that the hand piece is deviating from its desired path.

An alternative embodiment of some hardware components are shown in FIGS. 3G-I. Fiducial key 10′ has connection elements with suitable connecting portions to allow a tracking pole 11′ to position a tracking marker 12′ relative to the surgical site. Conceptually, fiducial key 10′ serves as an anchor for pole 11′ and tracking marker 12′ in much the same way as the earlier embodiment, although it has a distinct shape. The software of the monitoring system is pre-programmed with the configuration of each particularly identified fiducial key, tracking pole, and tracking marker, so that the location calculations are only changed according to the changed configuration parameters.

The materials of the hardware components may vary according to regulatory requirements and practical considerations. Generally, the key or fiducial component is made of generally radio opaque material such that it does not produce noise for the scan, yet creates recognizable contrast on the scanned image so that any identifying pattern associated with it may be recognized. In addition, because it is generally located on the patient, the material should be lightweight and suitable for connection to an apparatus on the patient. For example, in the dental surgery example, the materials of the fiducial key must be suitable for connection to a plastic splint and suitable for connection to a tracking pole. In the surgical example the materials of the fiducial key may be suitable for attachment to the skin or other particular tissue of a patient.

The vectorized tracking markers may be clearly identified by employing, for example without limitation, high contrast pattern engraving. The materials of the tracking markers are chosen to be capable of resisting damage in autoclave processes and are compatible with rigid, repeatable, and quick connection to a connector structure. The tracking markers and associated tracking poles have the ability to be accommodated at different locations for different surgery locations, and, like the fiducial keys, they should also be relatively lightweight as they will often be resting on or against the patient. The tracking poles must similarly be compatible with autoclave processes and have connectors of a form shared among tracking poles.

The tracker employed in tracking the fiducial keys, tracking poles and tracking markers should be capable of tracking with suitable accuracy objects of a size of the order of 1.5 square centimeters. The tracker may be, by way of example without limitation, a non-stereo camera, a stereo camera or stereo camera pair. While the tracker is generally connected by wire to a computing device to read the sensory input, it may optionally have wireless connectivity to transmit the sensory data to a computing device. In other embodiments, the tracker may be a non-stereo optical tracker.

In embodiments that additionally employ a trackable piece of instrumentation, such as a hand piece, vectorized tracking markers attached to such a trackable piece of instrumentation may also be light-weight; capable of operating in a 3 object array with a 90° relationship; optionally having a high contrast pattern engraving and a rigid, quick mounting mechanism to a standard hand piece.

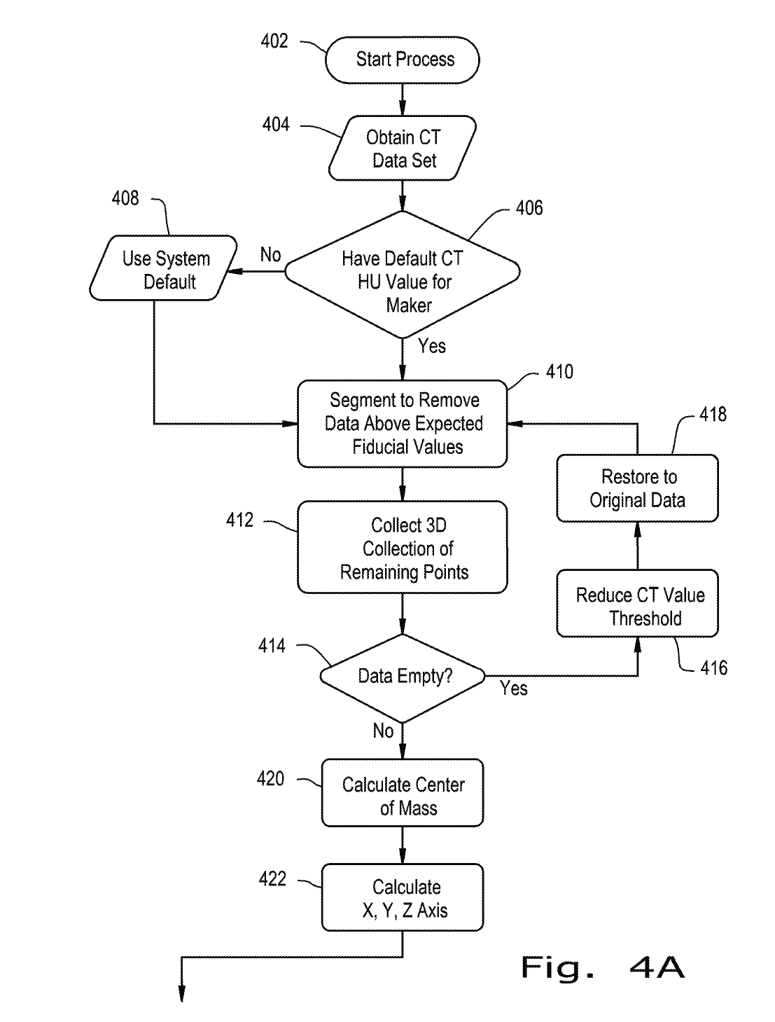

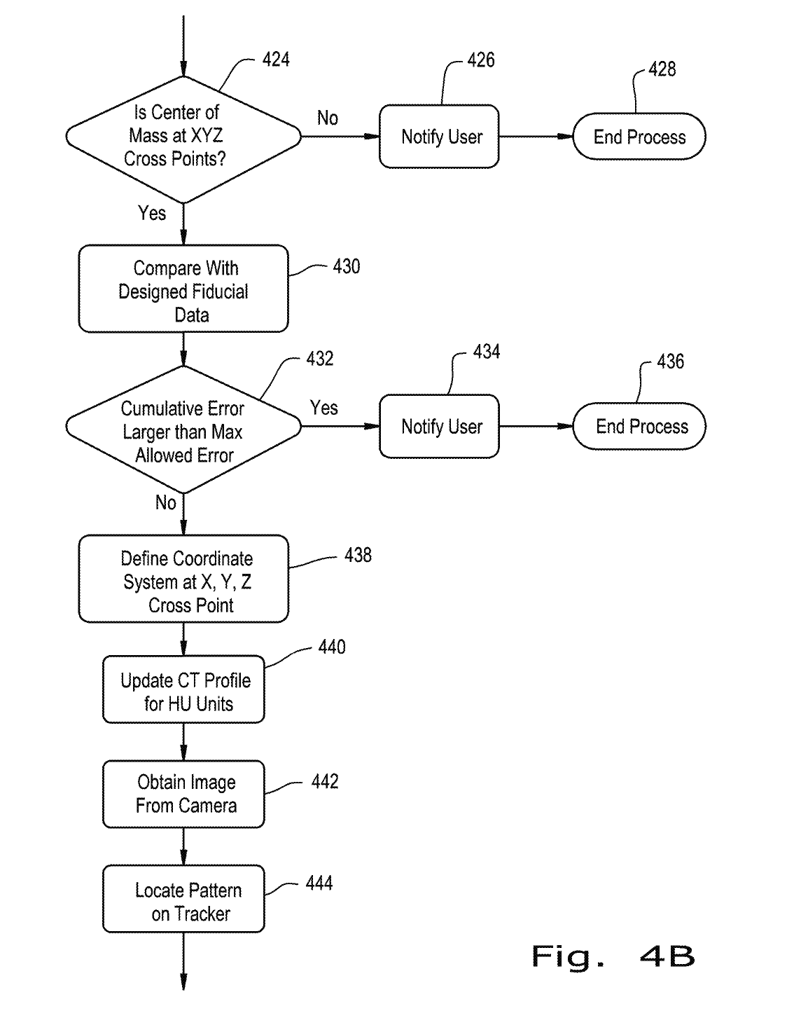

In another aspect there is presented an automatic registration method for tracking surgical activity, as illustrated in FIGS. 4A-C. FIG. 4A and FIG. 4B together present, without limitation, a flowchart of one method for determining the three-dimensional location and orientation of the fiducial reference from scan data. FIG. 4C presents a flow chart of a method for confirming the presence of a suitable tracking marker in image information obtained by the tracker and determining the three-dimensional location and orientation of the fiducial reference based on the image information.

Once the process starts [402], as described in FIGS. 4A and 4B, the system obtains a scan data set [404] from, for example, a CT scanner and checks for a default CT scan Hounsfield unit (HU) value [at 406] for the vectorized fiducial which may or may not have been provided with the scan based on a knowledge of the fiducial and the particular scanner model, and if such a threshold value is not present, then a generalized predetermined default value is employed [408]. Next the data is processed by removing scan segments with Hounsfield data values outside expected values associated with the fiducial key values [at 410], following the collection of the remaining points [at 412]. If the data is empty [at 414], the CT value threshold is adjusted [at 416], the original value restored [at 418], and the segmenting processing scan segments continues [at 410]. Otherwise, with the existing data a center of mass is calculated [at 420], along with calculating the X, Y, and Z axes [at 422]. If the center of mass is not at the cross point of the XYZ axes [at 424], then the user is notified [at 426] and the process stopped [at 428]. If the center of mass is at the XYZ cross point then the data points are compared with the designed fiducial data [430]. If the cumulative error is larger than the maximum allowed error [432] then the user is notified [at 434] and the process ends [at 436]. If not, then the coordinate system is defined at the XYZ cross point [at 438], and the scan profile is updated for the HU units [at 440].

Turning now to FIG. 4C, image information is obtained from the tracker, being a suitable camera or other sensor [442]. The image information is analyzed [444] to determine whether a tracking marker is present in the image information. If not, then the user is queried [446] as to whether the process should continue or not. If not, then the process is ended [448]. If the process is to continue, then the user may be notified [450] that no tracking marker has been found in the image information, and the process returns to obtaining image information [442]. If a tracking marker has been found based on the image information, or one has been attached by the user upon the above notification [at 450], the offset and relative orientation of the tracking marker to the fiducial reference is obtained [452] from a suitable database. The term “database” is used in this specification to describe any source, amount or arrangement of such information, whether organized into a formal multi-element or multi-dimensional database or not. Such a database may be stored, for example, in system memory 217, fixed disk 244, or in external memory through network interface 248. A single data set comprising offset value and relative orientation may suffice in a simple implementation of this embodiment of the invention and may be provided, for example, by the user or may be within a memory unit of the controller or in a separate database or memory.

The offset and relative orientation of the tracking marker is used to define the origin of a coordinate system at the fiducial reference and to determine the three-dimensional orientation of the fiducial reference based on the image information [454] and the registration process ends [458]. In order to monitor the location and orientation of the fiducial reference in real time, the process may be looped back from step [454] to obtain new image information from the camera [442]. A suitable query point may be included to allow the user to terminate the process. Detailed methods for determining orientations and locations of predetermined shapes or marked tracking markers from image data are known to practitioners of the art and will not be dwelt upon here. The coordinate system so derived is then used for tracking the motion of any items bearing vectorized tracking markers in the proximity of the surgical site. Other registration systems are also contemplated, for example using current other sensory data rather than the predetermined offset, or having a fiducial with a transmission capacity.

All vectorized tracking markers employed in the present invention may be passive. The term “passive” is used in the present specification to describe markers that do not rely on any own electronic, electrical, optoelectronic, optical, magnetic, wireless, inductive, or other active signaling function or on any incorporated electronic circuit, whether powered or unpowered, to be identified, located, or tracked. The term “own active signaling” is used in this specification to describe a signal that is temporally modulated by, on, or within the tracking marker. The tracking markers do not rely on motion, location, or orientation sensing devices, whether powered or unpowered, to be tracked. They cannot sense their own motion, location, or orientation, nor have they any ability to actively communicate. They bear distinctive markings and/or have distinctive shapes that allow them to be identified, located, and tracked in three dimensions by a separate tracker such as, for example without limitation, tracker 610 of FIG. 6 or tracker 508 of FIG. 5 described below, both as regards their location and as regards their orientation. In some embodiments, the tracker may be an optical tracker, more particularly, a non-stereo optical tracker. In other embodiments, the tracker may be a stereo tracker. Any one or more of identification, location, and tracking of the markers is solely on the basis of their distinctive markings and/or distinctive shapes. All fiducial references described in the present specification, may also be passive. This specifically includes fiducial references 10 and 10′ in FIGS. 3A to 3J, key or fiducial reference 502 of FIG. 5 and fiducial reference 602 of FIG. 6.

One example of an embodiment of the invention is shown in FIG. 5. In addition to passive vectorized fiducial key 502 mounted at a predetermined tooth and having a rigidly mounted passive vectorized tracking marker 504, an additional instrument or implement 506, for example a hand piece which may be a dental drill or scalpel, may be observed by a camera 508 serving as tracker of the monitoring system Implement 506 may bear a vectorized tracking marker 507 allowing it to be tracked by tracker 508. Tracker 508 may in some embodiments be, in particular, a non-stereo tracker. Tracker 508 supplies image information of a field of view of tracker 508 to controller 520, which displays derived information on a display system or monitor 530. Controller 520 may be based on, for example, processor 214 and memory 217 of computer 210 of FIG. 2 and monitor 530 may have with controller 520 the structural relation that display screen 224 has with central processor 214 in FIG. 2.

Another example of an embodiment of the invention is shown in FIG. 6. Surgery site 600, for example a human stomach or chest, may have fiducial key 602 fixed to a predetermined position to support tracking marker 604. Other apparatus with suitable tracking markers may be in use in the process of the surgery at surgery site 600. By way of non-limiting example, endoscope 606 may have a further passive vectorized tracking marker 607, and biopsy needle 608 may also be present at surgery site 600 bearing a passive vectorized tracking marker 609. Sensor 610, serving as tracker for the system, may be for example a camera, infrared sensing device, or RADAR. In particular, the tracker may be a two-dimensional imaging tracker that produces a two dimensional image of the surgery site 600 for use as image information for the purposes of embodiments of the invention, including two dimensional image information of any vectorized tracking markers in the field of view of the tracker. Sensor 610 may be, for example, a non-stereo optical camera. In other embodiments sensor 610 may be a stereo camera. Surgery site 600, endoscope 606, biopsy needle 608, fiducial key 602 and vectorized tracking markers 604, 607 and 609 may all be in the field of view of tracker 610. Sensor 610 supplies image information of a field of view of sensor 610 to controller 520 which displays derived information on a display system or monitor 530.

Trackers 508, 610 of the systems and methods of the embodiments of the present invention may comprise a single optical imager obtaining a two-dimensional image of the site being monitored. The system and method described in the present specification allow three-dimensional locations and orientations of tracking markers to be obtained using non-stereo-pair two-dimensional imagery. In some embodiments more than one imager may be employed as tracker, but the image information required and employed is nevertheless two-dimensional. Therefore, the two imagers may merely be employed to secure different perspective views of the site, each imager rendering a two-dimensional image that is not part of a stereo pair. This does not exclude the employment of stereo-imagers in obtaining the image information about the site, but the systems and methods of the present invention are not reliant on stereo imagery of the site in order to identify and track any of the passive vectorized tracking markers employed in the present invention. By virtue of their shapes or markings, the three-dimensional locations and orientations of the tracking markers may be completely determined from a single two-dimensional image of the field of view of the tracker.

In another aspect of the present invention there is therefore provided a method, described with reference to FIG. 5 and the flow chart in FIG. 7, for relating in real time the three-dimensional location and orientation of surgical site 550 on a patient to the location and orientation of the surgical site in a scan of surgical site 550, the method comprising removably and rigidly attaching [710] single passive scan-visible vectorized fiducial reference 502 to a fiducial location on the patient proximate surgical site 550; performing the scan with single fiducial reference 502 attached to the fiducial location to obtain [720] scan data; obtaining [730] the three-dimensional location and orientation of fiducial reference 502 from the scan data; obtaining [750] real time image information of surgical site 550 (using tracker 508); determining [760] in real time the three-dimensional location and orientation of single fiducial reference 502 from the image information; and deriving [770] a spatial transformation matrix or expressing in real time the three-dimensional location and orientation of fiducial reference 502 as determined from the image information in terms of the three-dimensional location and orientation of single fiducial reference 502 as determined from the scan data.

Obtaining [750] real time image information from surgical site 550 may comprise rigidly and removably attaching to fiducial reference 502 first passive vectorized tracking marker 504 in a fixed three-dimensional spatial relationship with fiducial reference 502, therewith disposing [740] tracking marker 504 in a field of view of tracker 508. First tracking marker 504 may be configured for having its location and its orientation determined based on the image information. Attaching first tracking marker 504 to single fiducial reference 502 may comprise rigidly and removably attaching first tracking marker 504 to the fiducial reference 502 by means of a tracking pole. In this regard, see for example tracking pole 11 of FIG. 3B used to attach vectorized tracking marker 12 to fiducial reference 10. Attaching first tracking marker 504 to single fiducial reference 502 may comprise rigidly and removably attaching to the fiducial reference 502 the tracking pole in a fixed three-dimensional spatial relationship with the fiducial reference 502, and the tracking pole may have a distinctly identifiable three-dimensional shape that allows its location and orientation to be uniquely determined from the image information.

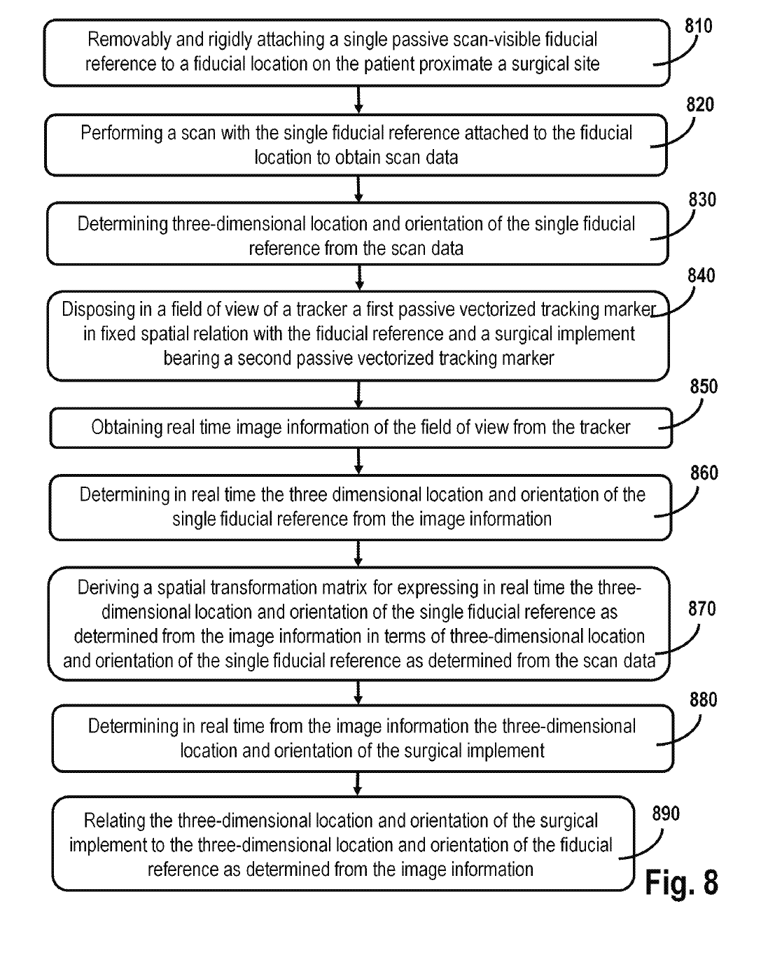

In yet a further aspect of the invention, explained at the hand of the flow chart of FIG. 8, there is provided a method for real time monitoring the position of an object, for example implement 506 in FIG. 5, in relation to surgical site 550 of a patient, the method comprising removably and rigidly attaching [810] single passive scan-visible fiducial reference 502 to a fiducial location on the patient proximate surgical site 550; performing [820] a scan with single fiducial reference 502 attached to the fiducial location to obtain scan data; determining [830] the three-dimensional location and orientation of single fiducial reference 502 from the scan data; obtaining [850] real time image information of surgical site 550 (using tracker 508); determining [860] in real time the three-dimensional location and orientation of single fiducial reference 502 from the image information; deriving [870] a spatial transformation matrix for expressing in real time the three-dimensional location and orientation of single fiducial reference 502 as determined from the image information in terms of the three-dimensional location and orientation of single fiducial reference 502 as determined from the scan data; determining [880] in real time the three-dimensional location and orientation of implement 506 from the image information; and relating [890] the three-dimensional location and orientation of implement 506 to the three-dimensional location and orientation of the fiducial reference 502 as determined from the image information.

Obtaining [850] of real time image information from surgical site 550 may comprise rigidly and removably attaching to fiducial reference 502 a first passive vectorized tracking marker 504 in a fixed three-dimensional spatial relationship with fiducial reference 502, therewith disposing [840] tracking marker 504 in a field of view of tracker 508. First tracking marker 504 may be configured for having its location and its orientation determined based on the image information. Attaching first tracking marker 504 to single fiducial reference 502 may comprise rigidly and removably attaching first tracking marker 504 to the fiducial reference 502 by means of a tracking pole. In this regard, see for example tracking pole 11 of FIG. 3B used to attach vectorized tracking marker 12 to fiducial reference 10. Attaching first tracking marker 504 to single fiducial reference 502 may comprise rigidly and removably attaching to the fiducial reference 502 the tracking pole in a fixed three-dimensional spatial relationship with the fiducial reference 502, and the tracking pole may have a distinctly identifiable three-dimensional shape that allows its location and orientation to be uniquely determined from the image information.

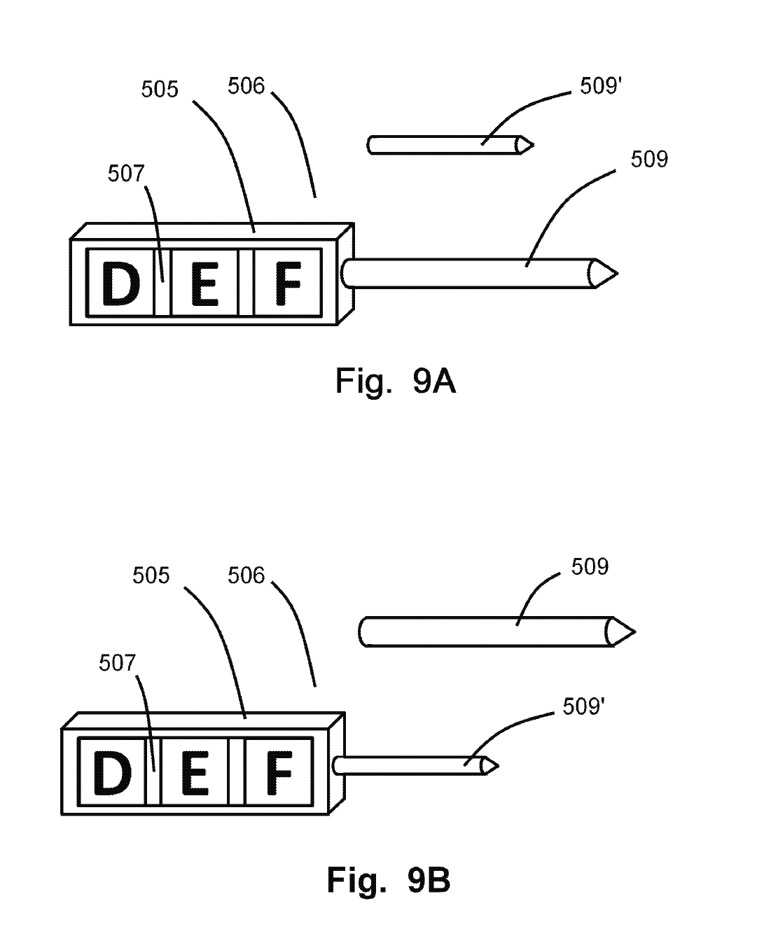

In some circumstances during surgery, surgical implements are changed or modified. An example is when one drill bit is exchanged for another. Certain embodiments of the invention are advantageous for the fact that the surgical navigation system may determine the characteristics of a drill bit inserted in the handpiece during the surgery. Specifying this manually to a software interface is inconvenient, interrupts workflow, and is error prone, while mechanically measuring the drill bit interrupts workflow and risks compromising the sterile part. Prior art optical methods require placing the surgical tool into a measuring device or in a known position against a target or reference. In a further aspect of the invention addressing this issue, surgical implement 506 of FIG. 5, shown in more detail in FIG. 9A and 9B, may comprise an invariant portion 505 and an interchangeable portion 509. For example, without limitation, implement 506 may be a dental drill 506 comprising a drill body 505 as invariant portion and an interchangeable drill bit 509 as interchangeable portion. In alternative embodiments, implement 506 may be a scalpel and the interchangeable portion 509 may be a scalpel blade. Drill bit 509 may, for example, be interchanged with drill bit 509′, which may, for example, have a different length and/or diameter. In other embodiments, interchangeable portions 509 and 509′ may be portions with different functions. The collective characteristic of all the interchangeable portions is that they are at least one of shapewise and dimensionally uniquely identifiable from the image information supplied by tracker 508 of FIG. 5. FIG. 9A schematically shows interchangeable portion 509 mounted in invariant portion 505, and FIG. 9B schematically shows interchangeable portion 509′ mounted in invariant portion 505. Invariant portion 505 is configured to allow interchangeable portions, for example interchangeable portions 509 and 509′, to be seated spatially consistently in invariant portion 505.

Tracker 508 of FIG. 5 is configured with a sufficiently high imaging resolution to resolve in the imaging information the dimensions and shapes of interchangeable portions 509 and 509′ in order for controller 520 to uniquely identify interchangeable portions 509 and 509′. To this end, the database of the monitoring system further comprises a data describing at least one of the dimensions, the shapes, and the configurations of all interchangeable portions of implement 506 available for use with the system. The database may be stored, for example, in system memory 217, fixed disk 244, or in external memory through network interface 248. The dimensions and shape of invariant portions, for example invariant portion 505 of FIGS. 9A and 9B, may also be stored in the database, so that the interchangeable portions, for example interchangeable portions 509 and 509′ of FIGS. 9A and 9B, may be uniquely identifiable from the image information.

In other embodiments, tracker 508 of FIG. 5 may have two imagers obtaining two separate perspectives of the field of view of tracker 508, 610 and the interchangeable portions 509 and 509′ in that field of view. This allows the working tip of interchangeable portions 509 and 509′ to be determined in three dimensions. From this may then be derived the length of the interchangeable portions 509 and 509′ so that the particular portion may then be identified from the database based on a unique length. In yet a further embodiment, the tip of the interchangeable portions 509 and 509′ located and oriented in this way may be related to the known and tracked three-dimensional location and orientation of tracking marker 507 on invariant portion 505 of implement 506. This relationship having been established, the three-dimensional location and orientation of the working tip of interchangeable portions 509 and 509′ may then be calculated in real time based on the tracked three-dimensional location and orientation of tracking marker 507.

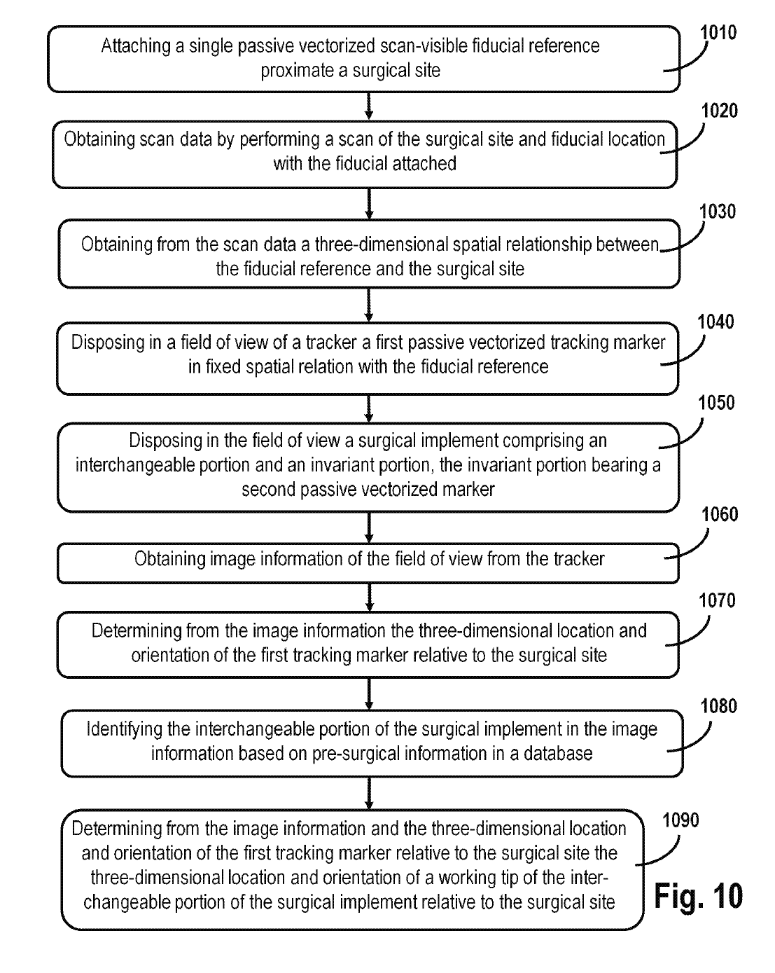

In a further aspect of the invention, described at the hand of FIG. 5 and FIG. 10, a method is provided for monitoring changes in a surgical implement 506 in three dimensions relative to a surgical site 550, the method comprising, attaching [1010] single passive vectorized scan-visible fiducial reference 502 at a fiducial location proximate surgical site 550; obtaining [1020] scan data by performing a scan of surgical site 550 and fiducial location with fiducial 502 attached; obtaining [1030] from the scan data a 3D spatial relationship between fiducial reference 502 and surgical site 550; disposing [1040] in a field of view of optical tracker 508 a first passive vectorized tracking marker 504 in fixed spatial relation with fiducial reference 502; disposing [1050] in the field of view surgical implement 506 comprising interchangeable portion 509 or 509′ and invariant portion 505, invariant portion 505 bearing a second passive vectorized marker 507; obtaining [1060] image information of the field of view from tracker 508; determining [1070] from the image information the 3D location & orientation of first tracking marker 504 relative to surgical site 550; identifying [1080] interchangeable portion 509, 509′ of surgical implement 506 in the image information; determining [1090] from the image information and from the 3D location and orientation of first tracking marker 504 relative to surgical site 550 the 3D location and orientation of a working tip of interchangeable portion 509, 509′ of surgical implement 506 relative to surgical site 550. Determining [1090] the location and orientation of a working tip of interchangeable portion 509, 509′ relative to surgical site 550 may further comprise identifying second tracking marker 507 in the image information and determining the three-dimensional location and orientation of second tracking marker 507 and invariant portion 505 attached to it.

In one embodiment of the method, identifying [1080] interchangeable portion 509, 509′ of surgical implement 506 in the image information is based on pre-surgical information in a database. In other embodiments of the method, identifying [1080] interchangeable portion 509, 509′ of surgical implement 506 in the image information comprises determining the three dimensional location of the working tip of interchangeable portion 509, 509′ and determining the length of interchangeable portion 509, 509′ from the three-dimensional location of the working tip and the three-dimensional location and orientation of second tracking marker 507 and invariant portion 505 attached to second tracking marker 507. Determining the three dimensional location of a working tip of interchangeable portion 509, 509′ comprises triangulating the three dimensional location of the working tip based on two separate perspectives of interchangeable portions 509, 509′ in the field of view of tracker 508.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A position monitoring system for a surgical procedure, the surgical procedure occurring on a surgical site on a surgical patient, the system comprising:
a single passive vectorized scan-visible fiducial reference adapted to be fixed to a fiducial location proximate the surgical site of the surgical patient;
a tracker configured to obtain image information within a field of view including at least a portion of the surgical site;
a first passive vectorized tracking marker removably disposed within the field of view in a predetermined fixed position and orientation relative to the fiducial reference; and
a controller having patient scan data from a scan of the surgical site and the fiducial reference when fixed to the fiducial location, the controller configured to receive the image information from the tracker and including a processor with memory and a software program having a series of instructions which when executed by the processor determines from the image information a current three-dimensional position and orientation of the first tracking marker, and relates the current position and orientation of the fiducial reference to the scan data based on the current three-dimensional position and orientation of the first tracking marker.

2. The position monitoring system of claim 1, wherein the tracker is a non-stereo optical tracker.

3. The position monitoring system of claim 1, further comprising a display system in communication with the controller, the display system adapted to show the current position and orientation of the fiducial reference relative to the patient scan data during the surgical procedure.

4. The position monitoring system of claim 1, wherein the fiducial reference comprises a material that is distinctly identifiable on an image from at least one of an X-ray, Magnetic Resonance Imaging (MRI), computerized tomography (CT), sonography, and cone beam computerized tomography (CBCT).

5. The position monitoring system of claim 1, wherein the fiducial reference has a distinct shape that allows its position and orientation to be determined from the patient scan data.

6. The position monitoring system of claim 1, wherein the fiducial reference has a label in a predetermined position such that the orientation of the fiducial reference is determinable from the patient scan data.

7. The position monitoring system of claim 1, wherein the fiducial reference is configured and arranged to fit a part of the patient being scanned.

8. The position monitoring system of claim 1, wherein the first tracking marker has a distinct sensible characteristic which is identifiable in the image information.

9. The position monitoring system of claim 1, wherein the first tracking marker has a distinct shape such that the location and orientation of the fiducial reference are determined from at least one image, including a sequence of images, from the tracker.

10. The position monitoring system of claim 1, wherein the first tracking marker is identifiable as a specific code determinable from the image information.

11. The position monitoring system of claim 10, wherein the specific code determines a specific marker of which a position and orientation relative to the fiducial reference is known and fixed.

12. The position monitoring system of claim 1, wherein the first tracking marker is adaptable in its physical arrangement to permit unobstructed access to the surgical site during different procedures or stages of a surgical procedure.

13. The position monitoring system of claim 1, further comprising a mounting arrangement between the fiducial reference and the first tracking marker, the mounting arrangement configured such that the first tracking marker is interchangeable with a second tracking marker to retain for the second tracking marker the predetermined fixed position and orientation of the first tracking marker relative to the fiducial reference.

14. The position monitoring system of claim 1, further comprising at least one further tracking marker, each at least one further marker being attached to an implement such that the position and orientation of the implement is determined by the software program.

15. The position monitoring system of claim 14, wherein the position and orientation of the implement is determined contemporaneously with the position and orientation of the fiducial reference.

16. The position monitoring system of claim 14, wherein each at least one further marker is individually distinct so that an identity of the attached implement is determinable from the image information.

17. The position monitoring system of claim 14, wherein the attached implement has an operating tip and the software program determines the position of the operating tip relative to the surgical site.

18. The position monitoring system of claim 17, wherein the operating tip is one of a drill bit of a dental drill, a sensor of an endoscope, and a blade of a scalpel.

19. The position monitoring system of claim 14, wherein at least one of the attached implements has multiple distinct identifiable tracking markers attached to different parts of the at least one implement such that at least one of the multiple distinct tracking markers is apparent to the tracker in any orientation of the at least one implement.

20. The position monitoring system of claim 14, wherein the implement is a dental drill.

21. The position monitoring system of claim 14, wherein the implement is an endoscope.

22. The position monitoring system of claim 14, wherein the implement is one of a biopsy needle and a surgical implant.

23. A system for monitoring in three dimensions relative to a surgical site changes in a surgical implement that comprises an interchangeable portion and an invariant portion, the system comprising:
a single passive vectorized scan-visible fiducial reference configured for attaching to a fiducial location proximate the surgical site;
a first passive vectorized tracking marker disposable in fixed spatial relation with the fiducial reference;
a second passive vectorized tracking marker rigidly attached to the invariant portion of the surgical instrument in a known location and orientation relative to the invariant portion;
a tracker disposed to obtain image information from a field of view including at least the first tracking marker and the surgical implement;
a controller having pre-surgical scan data from a scan of the surgical site and the fiducial location with the fiducial reference attached at the fiducial location, the controller configured to receive the image information from the tracker and including a processor with memory and a software program having a series of instructions which when executed by the processor determines from the image information a three-dimensional location and orientation of the first tracking marker relative to the surgical site, identifies the interchangeable portion of the surgical implement in the image information, and determines from the image information and from the three-dimensional location and orientation of the first tracking marker relative to the surgical site a three-dimensional location and orientation of a working tip of the interchangeable portion of the surgical implement relative to the surgical site.

24. The system of claim 23, wherein the software comprises a further series of instructions which when executed determines from the image information the three-dimensional location and orientation of the second tracking marker.

25. The system of claim 23, further comprising a database of pre-surgical information of the implement and wherein the software comprises a further series of instructions which when executed indentifies the interchangeable portion of the surgical implement in the image information based on the pre-surgical information in the database.

26. The system of claim 23, wherein the software comprises a further series of instructions which when executed determines the three dimensional location of the working tip of the interchangeable portion and a series of instructions which when executed determines the length of the interchangeable portion from the three-dimensional location of the working tip and the three-dimensional location and orientation of the second tracking marker and the invariant portion attached to the second tracking marker.

27. The system of claim 23, wherein the software comprises a further series of instructions which when executed triangulates the three dimensional location of the working tip based on two separate perspectives of the interchangeable portions in the field of view of the tracker.

28. The system of claim 23, wherein the tracker is a non-stereo optical tracker.

29. A method for relating in real time a three-dimensional location and orientation of a surgical site on a patient to a location and orientation of the surgical site in a scan of the surgical site, the method comprising:
attaching a single passive scan-visible vectorized fiducial reference to a fiducial location on the patient proximate the surgical site;
performing the scan with single fiducial reference attached to the fiducial location to obtain scan data;
obtaining the three-dimensional location and orientation of the fiducial reference from the scan data;
obtaining real time image information of the surgical site from a tracker;
determining in real time the three-dimensional location and orientation of the single fiducial reference from the image information; and
deriving a spatial transformation matrix for expressing in real time the three-dimensional location and orientation of the fiducial reference as determined from the image information in terms of the three-dimensional location and orientation of the single fiducial reference as determined from the scan data.

30. The method of claim 23, wherein obtaining the real time image information from the surgical site comprises disposing a first passive vectorized tracking marker in a field of view of the tracker by rigidly and removably attaching to the fiducial reference the first passive vectorized tracking marker in a fixed three-dimensional spatial relationship with the fiducial reference.

31. The method of claim 30, wherein attaching the first tracking marker to the single fiducial reference comprises rigidly and removably attaching the first tracking marker to the fiducial reference by means of a tracking pole.

32. The method of claim 31, wherein attaching the first tracking marker comprises rigidly and removably attaching to the fiducial reference the tracking pole in a fixed three-dimensional spatial relationship with the fiducial reference.

33. A method for real time monitoring the position of a surgical implement in relation to a surgical site of a patient, the method comprising:
attaching a single passive scan-visible fiducial reference to a fiducial location on the patient proximate the surgical site;
performing a scan with the single fiducial reference attached to the fiducial location to obtain scan data;
determining the three-dimensional location and orientation of single fiducial reference from the scan data;
obtaining real time image information of surgical site from a tracker;
determining in real time a three-dimensional location and orientation of the fiducial reference from the image information;
deriving a spatial transformation matrix for expressing in real time a three-dimensional location and orientation of the fiducial reference as determined from the image information in terms of the three-dimensional location and orientation of single fiducial reference as determined from the scan data;
determining in real time a three-dimensional location and orientation of the surgical implement from the image information; and
relating the three-dimensional location and orientation of the surgical implement to the three-dimensional location and orientation of the fiducial reference as determined from the image information.

34. The method of claim 33, wherein obtaining of the real time image information from the surgical site comprises disposing a first passive vectorized tracking marker in a field of view of the tracker by rigidly and removably attaching to the fiducial reference the first passive vectorized tracking marker in a fixed three-dimensional spatial relationship with the fiducial reference.

35. The method of claim 34, wherein attaching the first tracking marker to the single fiducial reference comprises rigidly and removably attaching the first tracking marker to the fiducial reference by means of a tracking pole.

36. The method of claim 35, wherein attaching the first tracking marker comprises rigidly and removably attaching to the fiducial reference the tracking pole in a fixed three-dimensional spatial relationship with the fiducial reference.

37. A method for monitoring changes in a surgical implement in three dimensions relative to a surgical site, the method comprising:
attaching a single passive vectorized scan-visible fiducial reference at a fiducial location proximate the surgical site;
obtaining scan data by performing a scan of the surgical site and the fiducial location with the fiducial reference attached;
obtaining from the scan data a three-dimensional spatial relationship between the fiducial reference and the surgical site;
disposing in a field of view of a tracker a first passive vectorized tracking marker in fixed spatial relation with the fiducial reference;
disposing in the field of view of the tracker the surgical implement comprising an interchangeable portion and an invariant portion, the invariant portion bearing a second passive vectorized marker;
obtaining image information of the field of view from the tracker;
determining from the image information a three-dimensional location and orientation of the first tracking marker relative to the surgical site;
identifying the interchangeable portion of the surgical implement in the image information; and
determining from the image information and from the three-dimensional location and orientation of the first tracking marker relative to the surgical site the three-dimensional location and orientation of a working tip of the interchangeable portion of the surgical implement relative to the surgical site.

38. The method of claim 37, wherein determining of the location and orientation of a working tip of the interchangeable portion relative to the surgical site comprises determining from the image information the three-dimensional location and orientation of the second tracking marker attached to the invariant portion.

39. The method of claim 37, wherein identifying the interchangeable portion of the surgical implement in the image information is based on pre-surgical information in a database.

40. The method of claim 37, wherein identifying the interchangeable portion of the surgical implement in the image information comprises determining the three dimensional location of the working tip of the interchangeable portion and determining the length of the interchangeable portion from the three-dimensional location of the working tip and the three-dimensional location and orientation of the second tracking marker and the invariant portion attached to the second tracking marker.

41. The method of claim 37, wherein determining the three dimensional location of a working tip of the interchangeable portion comprises triangulating the three dimensional location of the working tip based on two separate perspectives of the interchangeable portions \in the field of view of the tracker.
